(19) United States
(12) Patent Application Publication
(10) Pub. No.: US 2019/0135859 A1
(43) Pub. Date: May 9, 2019

(54) METHOD OF MANUFACTURING A PHARMACEUTICAL COMPOSITION

(71) Applicant: FERRING B.V., Hoofddorp (NL)

(72) Inventors: Raymond PETERSON, Boulder, CO (US); Robert HAMILTON, Boulder, CO (US); Lin CHEN, Boulder, CO (US); Mark OINEN, Boulder, CO (US)

(21) Appl. No.: 16/100,802

(22) Filed: Aug. 10, 2018

(30) Foreign Application Priority Data
Aug. 11, 2017 (EP) 17186049.7

(51) Int. Cl. C07K 1/16; C07K 1/30; C07K 7/16; C07K 7/06; B01D 9/00; B01D 15/16

(52) U.S. Cl. CPC C07K 1/16; C07K 1/306; C07K 7/16; A61P 5/10; B01D 9/0054; B01D 15/163; C07K 7/06

(57) ABSTRACT

A method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution.

The present invention relates to a method of manufacturing a pharmaceutical composition comprising oxytocin or an oxytocin receptor agonist, for example carbetocin.

### BACKGROUND

Carbetocin [also known as (1-desamino-1-monocarba-2(O-methyl)-tyrosine)oxytocin or 1-butanoic acid-2-(O-methy-L-tyrosine)-1-carbaoxytocin] is a long-acting synthetic oligopeptide analogue of oxytocin, with agonist action. Carbetocin incorporates the following replacements relative to oxytocin: a) the amino-group of cysteine (position 1) by a hydrogen atom; b) of its disulphide bond by a thioether bond; and c) of the hydroxyl group of tyrosine (position 2) by a methyloxyl group. Carbetocin (PABAL®, DURATOCIN®) is currently approved for the prevention of uterine atony following delivery of the infant by Caesarean section under epidural or spinal anaesthesia. The dosages used for this medical indication are relatively small, for instance of the order of 100 micrograms given once.

The synthesis of peptides may be carried using solid phase synthetic procedures, which are well known in the art. Solution phase synthesis is an alternative method which may be useful for small quantities of peptide. This stage of peptide production is known as the “upstream process”, and results in the formation of a crude peptide product.

Following the synthesis of the crude peptide, it is usually necessary to separate the peptide of interest from various peptide and non-peptide impurities. This step is known as the purification step.

Many methods of purifying peptides are known in the art. However, peptide purification methods typically include at least one chromatographic step, for example size exclusion chromatography, hydrophobic interaction chromatography, ion exchange chromatography, free-flow-electrophoresis, affinity chromatography, high performance liquid chromatography (HPLC) etc.

HPLC is a preferred method in the art. This form of chromatography involves the use of high pressure to force a solute through a column with a stationary phase. The most commonly employed form of HPLC is “reversed phase” HPLC. In “reversed phase” HPLC (also known as RP-HPLC) the column material is, or is bonded to hydrophobic material, and the peptides are eluted by a gradient of increasing amounts of an organic solvent, such as acetonitrile. The peptides elute according to their hydrophobicity. After purification by HPLC the peptide of interest is present in a solution which (other than the peptide of interest itself) only comprises volatile compounds.

### Isolation Step

Following the purification step, for example HPLC, for example “reversed phase” HPLC, the peptide typically has to be separated from volatile solvents. This step is known as the isolation step. Known methods of separating the peptide from the solvents include ultrafiltration and lyophilisation.

### Ultrafiltration

This method involves the use of selectively permeable membranes. The membrane is sufficiently permeable to allow solvent molecules (for example, water) through the membrane. However, the membrane is sufficiently impermeable so as to prevent the passage of the peptide through the membrane. The solution may be forced through the membrane by means of a mechanical pump, gas pressure or centrifugation.

### Lyophilisation

Lyophilisation (also known as freeze-drying) comprises a step of rapid freezing of the peptide-containing solution, typically by immersing a container holding the solution in liquid nitrogen. The container is subsequently placed in a vacuum chamber which comprises a cooling coil. The volatile solvents sublimate in the vacuum. The sublimation process ensures that the purified sample is kept cold.

Lyophilisation is the technique most commonly used in the art to isolate peptides from solution. This is principally because the technique is well known, reproducible and easy to carry out. Further, the stability of peptides is typically increased at low temperatures.

The purification and isolation steps, in combination, are known as the “downstream process”.

Methods for purification and isolation of carbetocin and related peptides are known in the art: CN104592362 describes a step of liquid chromatography purification of carbetocin followed by lyophilisation. In most cases the liquid chromatography step is HPLC. In WO2015185584 the purification and lyophilisation of oxytocin agonists other than carbetocin are described. Furthermore, CN102977192 describes a process of purifying carbetocin by combining liquid chromatography and ion exchange chromatography. After purification, the product goes through the step of desalination and lyophilisation. CN104744567 describes a process of purifying carbetocin by ion exchange chromatography followed by lyophilisation. CN101531705 describes a process of purifying carbetocin using reverse phase HPLC followed by transforming the product into an acetate salt using the ion exchange method. After being transformed into the salt, the product is subsequently isolated by lyophilisation.

It can be seen from the above references that there is a strong prejudice in the art towards using lyophilisation as the isolation step in the synthesis of carbetocin and other oxytocin receptor agonists.

However, there are several problems associated with lyophilisation, for example, a large amount of time has to be spent processing the peptide, and the cost of the refrigerant and equipment is very high. These problems may be acceptable when producing small amounts of a peptide. However, when mass producing the peptide, lyophilisation becomes a “bottleneck” in the production process.

CN106831951 discloses a method of manufacturing oxytocin by (1) concentrating a solution of crude oxytocin prepared by the method described in “China Pharmaceutical Industry, 2015, No. 1, the Preparation Method of Oxytocin;” (2) adding ethanol or isopropanol to the mixture, prior to cooling the mixture to −10 to 0° C. to form a precipitate; and (3) filtering and drying the precipitate. CN104650193 describes a method of manufacturing oxytocin by dissolving oxytocin in a mixed solvent system, evaporating the more volatile solvent and precipitating oxytocin from the remaining solvent. Solvent A may be acetone, methanol, or ethanol and solvent B may be acetonitrile or ethyl acetate.

As set out above, carbetocin is known (and approved) for use in the treatment of uterine atony. The dosages used for this medical indication are relatively small. Recently, however, there has been an increased need for oxytocin receptor agonists, e.g. carbetocin. For example, oxytocin receptors have recently been indicated in the treatment of Prader-Willi Syndrome (see WO2016/044131). Prader-Willi Syndrome is a genetic disorder characterised by hyperphagia, food seeking behaviour, rapid weight gain, compulsive behaviour and aggression in young children. As described in WO2016/044131, patients treated with carbetocin exhibit statistically significant improvement over placebo treated patients after 15 days in measurements of hyperphagia, obsessive compulsive disorder, food seeking behaviour and clinical global impression. A relatively large amount of the peptide has to be produced for this indication, because the dosages used are significantly higher than those used in the treatment of uterine atony, for instance of the order of tens of milligrams per day, and the treatment is more long term.

Accordingly, there exists a need in the art for an improved method of isolating oxytocin receptor agonists, e.g. carbetocin, to remove the lyophilisation “bottleneck”. Therefore, the present invention has amongst others the aim of providing a method of producing carbetocin which does not have any of the drawbacks from the known methods of production.

### SUMMARY

The present invention relates to a method of manufacturing a pharmaceutical composition, for example a pharmaceutical composition comprising oxytocin or an oxytocin receptor agonist, for example carbetocin.

In a first aspect, the present invention relates to a method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution.

In a second aspect, the present invention relates to a method of manufacturing oxytocin or oxytocin receptor agonist comprising the steps of:
i) synthesising or providing crude oxytocin or crude oxytocin receptor agonist;
ii) performing liquid chromatography, for example HPLC, for example reverse phase HPLC, on the crude oxytocin or crude oxytocin receptor agonist formed in step i) to provide a liquid chromatography solution comprising oxytocin or oxytocin receptor agonist;
iii) performing a step of solid-phase enrichment on the oxytocin or oxytocin receptor agonist formed in step ii) to provide a solid-phase enrichment solution comprising oxytocin or a oxytocin receptor agonist;
iv) distilling the solid-phase enrichment solution formed in step iii) to form dry solid oxytocin or dry solid oxytocin receptor agonist, or a distillate solution of oxytocin or oxytocin receptor agonist having a reduced content of a solvent of the oxytocin or oxytocin receptor agonist (e.g. water) relative to the original solution;
v) if present following step iv, redissolving the dry solid oxytocin or dry solid oxytocin receptor agonist to form a concentrated solution comprising oxytocin or a oxytocin receptor agonist having a reduced content of a solvent of the oxytocin or oxytocin receptor agonist (e.g. water) relative to the original solution; and
vi) combining an antisolvent with the distillate or concentrated solution comprising oxytocin or oxytocin receptor agonist having a reduced content of a solvent of the oxytocin or oxytocin receptor agonist (e.g. water) formed by step iv) or step v) so as to precipitate a product oxytocin or a product oxytocin receptor agonist from the solution.

### DETAILED DESCRIPTION OF THE INVENTION

The applicants have advantageously and surprisingly found that it is possible to isolate oxytocin agonist (e.g. carbetocin) without any requirement for lyophilisation by precipitation of oxytocin agonist (e.g. carbetocin) from solution using an antisolvent. The process of the invention may therefore avoid the lyophilisation bottleneck.

Accordingly, the present invention relates to a method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution.

The antisolvent may be a non-polar liquid, for example a liquid having a polarity index of less than or equal to 8, for example less than or equal to 7, for example less than or equal to 6, for example less than or equal to 5, for example less than or equal to 4. Preferably, the antisolvent has a polarity index greater than or equal to 2 and less than or equal to 5. The antisolvent may be an aprotic non-polar liquid, for example an ester or an ether, for example ethyl acetate (polarity index=4.4), diethyl ether (polarity index=2.8), ethyl tert-butyl ether, di-isopropyl ether (polarity index=2.2), 2-methyltetrahydrofuran, propyl acetate, isopropyl acetate, methyl propionate, ethyl propionate or methyl tert-butyl ether (polarity index=2.5). Methyl tert-butyl ether is the preferred antisolvent. Thus, in one embodiment, the antisolvent is methyl tert-butyl ether.

The term “polarity index” as used herein refers to the dimensionless Snyder polarity index as described by Snyder, L. R. “Classification of the Solvent Properties of Common Liquids,” Journal of Chromatography, vol. 92, 1974, 223-230. Polarity index is a measure of the relative interaction of a liquid with various polar test solutes. Polarity index increases with increasing polarity.

The solvent in the solution comprising oxytocin or oxytocin receptor agonist described herein may be a polar solvent. The solvent may be a solvent having a polarity index of at least 5, for example a mixture of a polar aprotic compound having a polarity index of at least 5 and a polar protic compound having a polarity index of at least 5.

The polar solvent may comprise a mixture of water (polarity index=9.0) and acetonitrile (polarity index=5.8). A preferred polar solvent is for example a mixture comprising 90% acetonitrile 10% water (v/v). A more preferred polar solvent is for example a mixture comprising between 93% acetonitrile 7% water (v/v) and 96% acetonitrile 4% water (v/v).

The solvent in the solution comprising oxytocin or oxytocin receptor agonist described herein may be any solvent in which the oxytocin or oxytocin receptor agonist is soluble in an amount at standard conditions of 0.01 mg/ml or greater, for example 0.05 mg/ml or greater, for example 0.1 mg/ml or greater, for example 0.5 mg/ml or greater, for example 1 mg/ml or greater, for example 5 mg/ml or greater, for example 10 mg/ml or greater, for example 20 mg/ml or greater.

The antisolvent may be a liquid in which the oxytocin or oxytocin receptor agonist dissolves less readily relative to a solvent, or in which the oxytocin or oxytocin receptor agonist is, relatively to a solvent, less soluble. The antisolvent may be selected relatively to the solvent and may be any solvent in which the oxytocin or oxytocin receptor agonist is soluble in an amount at standard conditions of less than 20 mg/ml, for example less than 10 mg/ml, for example less than 5 mg/ml, for example less than 1 mg/ml, for example less than 0.5 mg/ml, for example less than 0.1 mg/ml, for example less than 0.05 mg/ml, for example less than 0.01 mg/ml. It will be understood by the skilled person that when the oxytocin or oxytocin receptor agonist is soluble in an amount of, for example, 10 mg/ml or greater in a solvent, in an antisolvent it will be less soluble, i.e. it will be soluble in an amount of less than 10 mg/ml, for example less than 5 mg/ml, for example less than 1 mg/ml, for example less than 0.5 mg/ml, for example less than 0.1 mg/ml, for example less than 0.05 mg/ml, for example less than 0.01 mg/ml. Unless otherwise specified, the terms solvent and antisolvent refer to the solubility behaviour of the oxytocin or oxytocin receptor agonist at room temperature and atmospheric pressure.

Herein, the term “product” as in “product oxytocin” or “product oxytocin receptor agonist,” “product carbetocin” (that is, the product of the step of combining the solution with antisolvent) may have purity greater than or equal to 97.5%, for example greater than or equal to 98%, for example greater than or equal to 98.5%, for example greater than or equal to 99%, for example greater than or equal to 99.25%, for example greater than or equal to 99.5%.

The solution comprising oxytocin or oxytocin receptor agonist may comprise crude oxytocin or crude oxytocin receptor agonist. Herein, the term “crude” as in “crude oxytocin”, “crude oxytocin receptor agonist”, “crude carbetocin” etc. means oxytocin/oxytocin receptor agonist/carbetocin may have a purity of less than 97%, for example less than 96%, for example less than 95%, for example less than 92.5%, for example less than 90%.

The solution comprising oxytocin or oxytocin receptor agonist may comprise a mixture of acetonitrile and water. The solution comprising oxytocin or oxytocin receptor agonist may comprise acetonitrile and water, with a water content of lower than about 40% w/w, for example, lower than about 30% w/w, for example, lower than about 20% w/w, for example lower than about 15% w/w, for example lower than about 10% w/w, for example from about 5% w/w to about 8% w/w. Water contents within these ranges are preferred as solutions with higher water contents phase separate instead of causing precipitation. A solution of acetonitrile and water, with a water content of about 13% w/w or lower is preferred as this results in greater consistency in the purity of product oxytocin or product oxytocin receptor agonist between batches and a greater ability for the product oxytocin or product oxytocin receptor agonist to be analysed for residual solvent via gas chromatography. Most preferably, the solution comprising oxytocin or oxytocin receptor agonist comprises acetonitrile and water, with a water content of from about 5% w/w to about 8% w/w.

Oxytocin or oxytocin receptor agonist may be present in the solution comprising oxytocin or oxytocin receptor agonist at a concentration from about 25 g/L to about 45 g/L for precipitation to occur upon combination of the solution with the antisolvent. Preferably, oxytocin or oxytocin receptor agonist may be present in the solution at a concentration of about 28 g/L to about 40 g/L. Most preferably, oxytocin or oxytocin receptor agonist may be present in the solution at a concentration of about 35 g/L.

Instead of adding antisolvent to the solution, preferably the solution comprising oxytocin or oxytocin agonist may be added to the antisolvent.

It was found that adding the solution comprising oxytocin or oxytocin receptor agonist to the antisolvent quickly, for example over a period of 20 minutes or less, for example over a period of 15 minutes or less, for example over a period of 10 minutes or less, for example over a period of 7.5 minutes or less, for example over a period of 5 minutes or less results in the formation of smaller particles, which results in a greater consistency in the purity of product oxytocin or product oxytocin receptor agonist between batches and greater ability for the product oxytocin or oxytocin receptor agonist to be analysed for residual solvent via gas chromatography. Most preferred was addition over a period of 5 minutes or less. It was further found that use of methyl tert-butyl ether as the antisolvent limited the loss of the oxytocin or oxytocin receptor agonist in the mother liquor (i.e., the solution left after the precipitation) and washes to around 2%. In comparison, use of ethyl acetate as the antisolvent in comparison resulted in a loss of the oxytocin or oxytocin receptor agonist in mother liquors and washes to around 20%. Further precipitation in methyl tert-butyl ether was found to remove an impurity at relative retention time (RRT) 0.63 which was present when precipitation was carried out in other antisolvents. Relative retention time (RRT) refers to the retention time of the analyte impurity relative to the retention time of oxytocin or oxytocin receptor agonist as measured by analytical HPLC. Analytical HPLC was carried out as described herein with respect to Example 1, step ii) in the Example section below.

Preferably, the solution comprising oxytocin or oxytocin receptor agonist may be added to 2 to 20 volume equivalents of the antisolvent, for instance 5 volume equivalents of antisolvent.

Prior to the addition of solution the antisolvent, the antisolvent may be at a temperature lower than ambient temperature, for example at a temperature of from −40° C. to 10° C., preferably at a temperature of from −20° C. to 0° C. The precipitated product may be agitated, such as by stirring, while aging for several hours, for instance 3 hours. Preferably, the precipitated product may be aged at a temperature of from 0° C. to 60° C., most preferably at a temperature of from 15° C. to 30° C.

The precipitated product may be agitated, such as by stirring, while aging for several hours, for instance 3 hours. Preferably, the precipitated product may be aged at a temperature of from 0° C. to 60° C., most preferably at a temperature of from 15° C. to 30° C.

In a most preferred embodiment, the present invention relates to a method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution, wherein the antisolvent is methyl tert-butyl ether, the solvent in the solution is a mixture of acetonitrile and water, the water content is between 5 to 13% w/w and the oxytocin or oxytocin receptor agonist is present in the solution at a concentration of approximately 25 to 45 mg/ml. Most preferably, the oxytocin receptor agonist is carbetocin. However, in further/other embodiments, the oxytocin receptor agonist may be another compound, as set out below.

In another preferred embodiment, the method of manufacturing oxytocin or oxytocin receptor agonist comprises the steps of:
i) synthesising or providing crude oxytocin or crude oxytocin receptor agonist;
ii) performing liquid chromatography, for example HPLC, for example reverse phase HPLC, on the crude oxytocin or crude oxytocin receptor agonist formed in step i) to provide a liquid chromatography solution comprising oxytocin or a oxytocin receptor agonist;
iii) performing a step of solid-phase enrichment on the oxytocin or oxytocin receptor agonist formed in step ii) to provide a solid-phase enrichment solution comprising oxytocin or a oxytocin receptor agonist;
iv) distilling the solid-phase enrichment solution formed in step iii) to form dry solid oxytocin or dry solid oxytocin receptor agonist or a distillate solution of oxytocin or oxytocin receptor agonist having a reduced content of a solvent of the oxytocin or oxytocin receptor agonist (e.g. water) relative to the original solution;
v) if present following step iv, redissolving the dry solid oxytocin or dry solid oxytocin receptor agonist to form a concentrated solution comprising oxytocin or a oxytocin receptor agonist having a reduced content of a solvent of the oxytocin or oxytocin receptor agonist (e.g. water) relative to the original solution; and
vi) combining an antisolvent with the distillate or concentrated solution comprising oxytocin or oxytocin receptor agonist having a reduced content of a solvent of the oxytocin or oxytocin receptor agonist (e.g. water) formed by step iv) or step v) so as to precipitate a product oxytocin or a product oxytocin receptor agonist from the solution.

In this preferred embodiment, the steps of the method are performed in the order listed above. Optional and preferred features of this embodiment are as set out further under the respective headings “Step i” to “Step vi” in the description below.

The method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution may optionally comprise a further step of providing or synthesising the oxytocin or oxytocin receptor agonist, prior to the step of combining the antisolvent with the solution comprising oxytocin or oxytocin receptor agonist so as to precipitate the product oxytocin or a product oxytocin receptor agonist from the solution. Optional and preferred features of this step are set out further under the heading “Step i)” below.

The method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution may optionally comprise a further step of performing liquid chromatography on oxytocin or oxytocin receptor agonist to form a liquid chromatography solution, prior to the step of combining an antisolvent with a solution comprising oxytocin or oxytocin receptor agonist so as to precipitate a product oxytocin or a product oxytocin receptor agonist from the solution. The liquid chromatography step may also occur after oxytocin or oxytocin agonist is synthesised or provided. Optional and preferred features of the liquid chromatography step are set out further under heading “Step ii)” below.

The method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution may optionally comprise a further step of performing solid-phase enrichment on oxytocin or oxytocin receptor agonist to form a solid-phase enrichment solution comprising oxytocin or oxytocin receptor agonist, prior to the step of combining an antisolvent with a solution comprising oxytocin or oxytocin receptor agonist so as to precipitate a product oxytocin or a product oxytocin receptor agonist from the solution. The solid-phase enrichment step may also occur after the liquid chromatography step, after the oxytocin or oxytocin receptor agonist is synthesised or provided in the absence of a liquid chromatography step, or after both the liquid chromatography step and the synthesis or provision step. Optional and preferred features of the solid-phase enrichment step are set out further under heading “Step iii)” below.

The method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution may optionally comprise a further step of distilling a solution of oxytocin or oxytocin receptor agonist to form dry solid oxytocin or oxytocin receptor agonist or a solution having a reduced content of the solvent of the oxytocin or oxytocin receptor agonist, (for example water) relative to the original solution, prior to the step of combining an antisolvent with the solution comprising oxytocin or oxytocin receptor agonist having a reduced content of the solvent of the oxytocin or oxytocin receptor agonist (if present) so as to precipitate a product oxytocin or a product oxytocin receptor agonist from the solution having a reduced content of the solvent of the oxytocin or oxytocin receptor agonist (if present). The distillation step may also occur after the solid-phase enrichment step in the absence of a liquid chromatography step, after the liquid chromatography step in the absence of a solid-phase enrichment step, after the synthesis or provision step with an intervening liquid chromatography step being present and an intervening solid phase enrichment step being absent, after the synthesis or provision step with an intervening liquid chromatography step being absent and an intervening solid-phase enrichment step being present, after the synthesis or provision step with an intervening liquid chromatography step and an intervening solid phase enrichment step both being present, or both being absent. Optional and preferred features of the distillation step are set out further under heading “Step iv)” below. By intervening it is meant that the step or steps occur(s) between two specified steps. Where two or more steps are referred to as intervening steps, the order in which the intervening steps are listed is preferred, but no specific order among the intervening steps is required.

Where the distillation step results in the formation of dry solid oxytocin or oxytocin receptor agonist, the method may further comprise re-dissolving the solid oxytocin or solid oxytocin receptor agonist to form a solution comprising oxytocin or a oxytocin receptor agonist having a reduced content of a solvent of the oxytocin or oxytocin receptor agonist (e.g. water) relative to the original solution, prior to the step of combining an antisolvent with the solution comprising oxytocin or oxytocin receptor agonist having a reduced content of the solvent of the oxytocin or oxytocin receptor agonist so as to precipitate a product oxytocin or a product oxytocin receptor agonist from the solution having a reduced content of the solvent of the oxytocin or oxytocin receptor agonist. The re-dissolution step, if necessary, occurs immediately after the distillation step, with all other features of the distillation step being as referred to above. Optional and preferred features of the re-dissolution step are as set out further under heading “Step v)” below.

Step i: Synthesising or Providing Oxytocin or Oxytocin Receptor Agonist

Oxytocin or oxytocin receptor agonist may be synthesised by methods well known in the art, for example the methods described in WO2009/122285 (International Patent Application No. PCT/IB2009/005351) of Ferring B.V., or analogous method.

The provided or synthesised oxytocin or oxytocin receptor agonist may comprise crude oxytocin or crude oxytocin receptor agonist or may be in the form of a solution comprising oxytocin or oxytocin receptor agonist. The provided or synthesised oxytocin or oxytocin receptor agonist may be in the form of a solution comprising crude oxytocin or oxytocin receptor agonist.

Step ii: Liquid Chromatography

The step of performing liquid chromatography (step ii above), for example HPLC, for example Reverse Phase HPLC may comprise the use of a column with a stationary phase together with a mobile phase. The mobile phase may comprise a first liquid and/or a second liquid.

The oxytocin or oxytocin receptor agonist may comprise crude oxytocin or crude oxytocin receptor agonist or may be in the form of a solution comprising oxytocin or oxytocin receptor agonist. The oxytocin or oxytocin receptor agonist may be in the form of a solution comprising crude oxytocin or oxytocin receptor agonist. The oxytocin or oxytocin receptor agonist may be dissolved in a mobile phase and loaded onto a liquid chromatography column with a stationary phase and may be separated from impurities in step ii) on the basis of differing retention times on the stationary phase of the liquid chromatography column.

The first liquid may comprise a solution (e.g. aqueous solution) comprising a solvent and a solute. The solute may be a buffer. The buffer may be, for example, an ammonium salt (for example ammonium acetate and/or ammonium hydroxide) or acetic acid. The solvent may be any solvent in which the oxytocin or oxytocin receptor agonist is soluble in an amount at standard conditions of 0.01 mg/ml or greater, for example 0.05 mg/ml or greater, for example 0.1 mg/ml or greater, for example 0.5 mg/ml or greater, for example 1 mg/ml or greater, for example 5 mg/ml or greater, for example 10 mg/ml or greater, for example 20 mg/ml or greater. The solvent is preferably a hydrophilic solvent. Preferably the (hydrophilic) solvent has a polarity index of greater or equal to 5, greater than equal to 6, greater than or equal to 7, or greater than or equal to 8. Most preferably the hydrophilic solvent is water (polarity index=9.0).

The second liquid may be or comprise a hydrophobic liquid. Preferably, the polarity index of the hydrophobic liquid is less than 8 for example from 4 to 8, for example from 4.5 to 7.5. The polarity index of the hydrophobic liquid may be less than 7, for example from 5 to 7, for example from 5.5 to 6.5. The polarity index of the hydrophobic liquid may be less than 6. Preferably, the hydrophobic liquid is acetonitrile (polarity index=5.8), methanol (polarity index=5.1) or ethanol (polarity index=5.2). Most preferably the hydrophobic liquid is methanol.

The first liquid may comprise 1 mM to 10 M ammonium salt, for example from 2 mM to 5 M ammonium salt, for example from 5 mM to 2M ammonium salt, for example from 10 mM to 1M ammonium salt, for example from 20 mM to 500 mM ammonium salt, for example from 50 mM to 200 mM ammonium salt, for example about 100 mM ammonium salt. The first liquid may comprise from 0.001% to 50% acetic acid v/v, for example from 0.002% to 20% acetic acid v/v, for example from 0.005% to 10% acetic acid v/v, for example from 0.01% to 5% acetic acid v/v, for example from 0.02% to 2% acetic acid v/v, for example from 0.05% to 1% acetic acid v/v, for example from 0.1% to 0.5% acetic acid v/v, for example about 0.2% acetic acid v/v. Preferably, the first liquid comprises from 20 mM to 500 mM, and most preferably from 50 mM to 200 mM, aqueous ammonium acetate. The second liquid may comprise 50% v/v or more methanol, for example 60% v/v or more methanol, for example 70% v/v or more methanol, for example 80% v/v or more methanol, for example 90% v/v or more methanol, for example 95% v/v or more methanol, for example 98% v/v or more methanol, for example 99% v/v or more methanol, for example substantially pure methanol, for example pure methanol, i.e. 100% v/v methanol. Most preferably, the second liquid comprises pure methanol.

The mobile phase initially added to the column in step ii) may comprise greater or equal to 50% (v/v) of the first liquid, for example greater than or equal to 60% (v/v) of the first liquid, for example greater than or equal to 70% (v/v) of the first liquid, for example greater than or equal to 80% (v/v) of the first liquid, for example greater than or equal to 90% (v/v) of the first liquid, for example greater than or equal to 92.5% (v/v) of the first liquid, for example 92.5% to 99% (v/v) of the first liquid. For example, and preferably, 93% to 98% (v/v) of the first liquid, for example, and most preferably, about 95% (v/v) of the first liquid. Preferably, the mobile phase initially added to the column also comprises the second liquid, for example the mobile phase initially added to the column may comprise less than 50% (v/v) of the second liquid, for example less than 40% (v/v) of the second liquid, for example less than 30% (v/v) of the second liquid, for example less than 20% (v/v) of the second liquid, for example less than 10% (v/v) of the second liquid, for example for example less than 1% to 7.5% (v/v) of the second liquid, for example, and preferably, less than 2% to 7% (v/v) of the second liquid, for example, and most preferably, about 5% (v/v) of the second liquid.

The column used step ii) may be a C-18 preparative column, for example a Kromasil™ C-18 100-10 or Phenomenex™ Luna C18(2).

The amount (v/v) of the first liquid in the mobile phase may subsequently be decreased. The decrease may be a gradual decrease or an abrupt decrease. Preferably, however, the decrease comprises an initial decrease portion, followed by a plateau, followed by a subsequent decrease portion. Preferably, the initial decrease portion is a gradual decrease portion and the subsequent decrease portion is an abrupt decrease portion. An abrupt change may mean a rate of change of about 20% (v/v) per minute or greater, for example greater than about 50% (v/v) per minute. A gradual change means a rate of change of from 0.5% (v/v) to less than 20% (v/v) per minute, for example to less than about 10% per minute, for example to less than about 5% per minute. A plateau means a rate of change from 0% (v/v) to less than 0.5% (v/v) per minute, for at least 2 minutes.

The amount (v/v) of the first liquid in the mobile phase may be decreased to less than 50% (v/v) of the mobile phase, for example less than 40% (v/v) of the mobile phase, for example less than 30% (v/v) of the mobile phase, for example less than 20% (v/v) of the mobile phase, for example less than 10% (v/v) of the mobile phase, for example less than 1% to 7.5% (v/v) of the mobile phase, for example and preferably less than 2% to 7% (v/v) of the mobile phase, for example and most preferably about 5% (v/v) of the mobile phase.

The amount (v/v) of the second liquid in the mobile phase may be increased, for example a gradual increase or an abrupt increase. Preferably, however, the increase comprises an initial increase portion, followed by a plateau, followed by a subsequent increase portion. Preferably, the initial increase portion is a gradual increase portion and the subsequent increase portion is an abrupt increase portion. Preferably any increase portion in the amount (v/v) of the second liquid in the mobile phase is simultaneous with any decrease portion in the amount (v/v) of the first liquid in the mobile phase. The amount of the second liquid in the mobile phase may be increased to greater than or equal to 50% (v/v) of the mobile phase, for example greater than or equal to 60% (v/v) of the mobile phase, for example greater than or equal to 70% (v/v) of the mobile phase, for example greater than or equal to 80% (v/v) of the mobile phase, for example greater than or equal to 90% (v/v) of the mobile phase, for example 92.5% to 99% (v/v) of the mobile phase, for example and preferably 93% to 98% (v/v) of the mobile phase, for example and most preferably, about 95% of the mobile phase.

Following the decrease in the amount (v/v) of the first liquid in the mobile phase and/or the increase in the amount (v/v) of the second liquid in the mobile phase, the amount (v/v) of the first liquid in the mobile phase may be increased and/or the amount (v/v) of the second liquid in the mobile phase may be decreased.

The increase in the amount (v/v) of the first liquid in the mobile phase may be a gradual increase or an abrupt increase. Preferably, the increase in the amount (v/v) of the first liquid in the mobile phase is an abrupt increase. The amount of the first liquid in the mobile phase may be increased to greater than or equal to 50% (v/v) of the mobile phase, for example greater than or equal to 60% (v/v) of the mobile phase, for example greater than or equal to 70% (v/v) of the mobile phase, for example greater than or equal to 80% (v/v) of the mobile phase, for example greater than or equal to 90% (v/v) of the mobile phase, for example 92.5% to 99% (v/v) of the mobile phase, for example and preferably 93% to 98% (v/v) of the mobile phase, for example, and most preferably about 95% of the mobile phase.

The decrease in the amount (v/v) of the second liquid in the mobile phase may be a gradual decrease or an abrupt decrease. Preferably this decrease is an abrupt decrease, simultaneous with the increase in the amount (v/v) of the first liquid in the mobile phase.

The amount (v/v) of the second liquid in the mobile phase may be decreased to less than 50% (v/v) of the mobile phase, for example less than 40% (v/v) of the mobile phase, for example less than 30% (v/v) of the mobile phase, for example less than 20% (v/v) of the mobile phase, for example less than 10% (v/v) of the mobile phase, for example less than 1% to 7.5% (v/v) of the mobile phase, for example and preferably less than 2% to 7% (v/v) of the mobile phase, for example and most preferably about 5% (v/v) of the mobile phase.

The flow rate in the column may be from around 0.1 mL/min to around 100 mL/min, for example from around 0.5 mL/min to around 50 mL/min, for example from around 1 mL/min to around 20 mL/min, for example from around 1 mL/min to around 10 mL/min, for example from around 2 mL/min to around 5 mL/min. Preferably, the flow rate in the column may be from around 2 mL/min to around 5 mL/min.

In a preferred embodiment, the first liquid comprises an aqueous ammonium acetate buffer from 50 mM to 200 mM. The second liquid comprises pure methanol. The mobile phase initially added to the column most preferably comprises about 95% (v/v) of the first liquid and about 5% (v/v) of the second liquid. Preferably, the amount (v/v) of the first liquid in the mobile phase may subsequently be decreased, with an initial decrease portion that is a gradual decrease portion, a plateau, and a subsequent decrease portion that is an abrupt decrease portion. The amount (v/v) of the first liquid is preferably decreased to about 5% (v/v) of the mobile phase. Preferably, the amount (v/v) of the second liquid in the mobile phase may be increased, and any increase portion in the amount (v/v) of the second liquid in the mobile phase is simultaneous with any decrease portion in the amount (v/v) of the first liquid in the mobile phase. Preferably the amount (v/v) of the second liquid is increased to about 95% (v/v) of the mobile phase. Preferably, the initial increase portion in the amount of the second liquid is a gradual increase portion, followed by a plateau, and a subsequent increase portion that is an abrupt increase portion.

Preferably, following the decrease in the amount (v/v) of the first liquid in the mobile phase and the increase in the amount (v/v) of the second liquid in the mobile phase, the amount (v/v) of the first liquid in the mobile phase is increased and the amount (v/v) of the second liquid in the mobile phase is decreased. Preferably, the increase in the amount (v/v) of the first liquid in the mobile phase is an abrupt increase and the decrease in the amount (v/v) of the second liquid is an abrupt decrease, simultaneous with the increase in the amount (v/v) of the first liquid in the mobile phase. Preferably the amount (v/v) of the first liquid is increased to about 95% (v/v) of the mobile phase and the amount (v/v) of the second liquid is decreased to about 5% (v/v) of the mobile phase. Preferably, the flow rate in the column may be from around 2 mL/min to around 5 mL/min.

The UV absorbance (λ=220 nm) of the mobile phase may be detected with a UV detector and plotted against retention time on the column to produce a liquid chromatography chromatogram. The column fractions may be collected and the oxytocin or oxytocin receptor agonist may be separated from impurities on the basis of differing retention times on the liquid chromatography column. The eluent of the major peak may be isolated as a pooled purification fraction from the mobile phase. The pooled purification fraction will comprise fractions that are accepted for pooling because they meet the specifications for the final API for purity and impurity limits. Hereinafter, the pooled purification fraction of the mobile phase corresponding to the major peak in the liquid chromatography chromatogram (UV absorbance, λ=220 nm) and meeting API specifications is referred to as the “liquid chromatography solution.”

Subsequently, analytical liquid chromatography may be performed to assess the purity of the oxytocin or oxytocin receptor agonist in the liquid chromatography solution. The integrated area under the major peak in the analytical chromatogram (UV absorbance, λ=220 nm) will be proportional to the concentration of the oxytocin or oxytocin receptor agonist, and the integrated area under the minor peaks in the chromatogram will be proportional to the concentrations of impurities, such that a purity of oxytocin or oxytocin receptor agonist may be determined.

Step iii: Solid Phase Enrichment

Solid phase enrichment (step iii)) is well known in the art. The step of solid phase enrichment (step iii) may comprise the use of a third liquid and/or a fourth liquid.

The oxytocin or oxytocin receptor agonist may comprise crude oxytocin or crude oxytocin receptor agonist or may be in the form of a solution comprising oxytocin or oxytocin receptor agonist. The oxytocin or oxytocin receptor agonist may be in the form of a solution comprising crude oxytocin or oxytocin receptor agonist. The oxytocin or oxytocin receptor agonist may be dissolved in a mobile phase and loaded onto a solid-phase enrichment column with a stationary phase, such that solid-phase enrichment occurs in step iii) on the basis of differing retention times on the stationary phase of the solid-phase enrichment column.

The third liquid may be or comprise a solvent of oxytocin or oxytocin agonist receptor (e.g. water). The solvent of oxytocin or oxytocin receptor agonist may be any solvent in which the oxytocin or oxytocin receptor agonist is soluble in an amount at standard conditions of 0.01 mg/ml or greater, for example 0.05 mg/ml or greater, for example 0.1 mg/ml or greater, for example 0.5 mg/ml or greater, for example 1 mg/ml or greater, for example 5 mg/ml or greater, for example 10 mg/ml or greater, for example 20 mg/ml or greater. The third liquid is preferably a hydrophilic solvent. Preferably the (hydrophilic) solvent has a polarity index of greater or equal to 5, greater than equal to 6, greater than or equal to 7, or greater than or equal to 8. Most preferably the hydrophilic solvent is water (polarity index=9.0).

The fourth liquid may be or comprise a hydrophobic liquid. Preferably the polarity index of the hydrophobic liquid is less than 8, for example from 4 to 8, for example from 4.5 to 7.5. The polarity index of the hydrophobic liquid may be less than 7, for example from 5 to 7, for example from 5.5 to 6.5. The polarity index of the hydrophobic liquid may be less than 6. Preferably the hydrophobic liquid is acetonitrile (polarity index=5.8), methanol (polarity index=5.1) or ethanol (polarity index=5.2). Preferably the hydrophobic liquid is acetonitrile. The step of solid phase enrichment may result in an eluent having a water content of around 50-60% (w/w).

The concentration of oxytocin or oxytocin receptor agonist in the eluent may be around 2 g/L to 100 g/L, for example from around 5 g/L to 50 g/L, for example from 10 g/L to 30 g/L, for example from 15 g/L to 25 g/L, for example around 20 g/L, for example around 27.5 g/L to around 50 g/L, for example around 33 g/L to around 38 g/L.

The mobile phase initially added to the column in step iii) may comprise greater or equal to 50% (v/v) of the third liquid, for example greater than or equal to 60% (v/v) of the third liquid, for example greater than or equal to 70% (v/v) of the third liquid, for example greater than or equal to 80% (v/v) of the third liquid, for example and preferably greater than or equal to 90% (v/v) of the third liquid.

The column used in step iii) may be a polystyrene based chromatography support, for example an Amberchrom™ CG300 resin, such as Amberchrom™ CG300M. Alternatively, the column used in step iii) may be an Amberlite™ XAD resin or a Purolite™ resin.

The amount (v/v) of the third liquid in the mobile phase may be decreased to less than 90% (v/v) of the mobile phase, for example less than 80% (v/v) of the mobile phase, for example from 10% to 90% (v/v) of the mobile phase, for example from 20% to 80% (v/v) of the mobile phase, for example from 25% to 75% (v/v) of the mobile phase, for example from 30% to 70% (v/v) of the mobile phase, for example from 35% to 65% (v/v) of the mobile phase, for example from 40% to 60% (v/v) of the mobile phase, for example and preferably from 45% to 55% (v/v) of the mobile phase, for example and most preferably about 50% (v/v) of the mobile phase.

The decrease in the amount (v/v) of the third liquid in the mobile phase may be a gradual decrease or an abrupt decrease. Preferably the decrease is an abrupt decrease. Preferably the abrupt decrease is followed by a plateau.

The mobile phase initially added to the column in step iii) may comprise less than 50% (v/v) of the fourth liquid, for example less than 40% (v/v) of the fourth liquid, for example less than 30% (v/v) of the fourth liquid, for example less than 20% (v/v) of the fourth liquid, for example and preferably less than 10% (v/v) of the fourth liquid.

The amount (v/v) of the fourth liquid in the mobile phase may be increased, for example a gradual increase or an abrupt increase. Preferably this increase is simultaneous with the decrease in the amount (v/v) of the third liquid in the mobile phase. Preferably the abrupt increase is followed by a plateau.

An abrupt change may mean a rate of change of greater than about 20% per minute, for example greater than about 50% per minute. A gradual change means a rate of change of less than 20% per minute, for example less than about 10% per minute, for example less than about 5% per minute. A plateau means a rate of change from 0% (v/v) to less than 0.5% (v/v) per minute, for at least 2 minutes.

The amount (v/v) of the fourth liquid in the mobile phase may be increased to greater than 10% (v/v) of the mobile phase, for example greater than 20% (v/v) of the mobile phase, for example from 10% to 90% (v/v) of the mobile phase, for example from 20% to 80% (v/v) of the mobile phase, for example from 25% to 75% (v/v) of the mobile phase, for example from 30% to 70% (v/v) of the mobile phase, for example from 35% to 65% (v/v) of the mobile phase, for example from 40% to 60% (v/v) of the mobile phase, for example and preferably from 45% to 55% (v/v) of the mobile phase, for example and most preferably about 50% (v/v) of the mobile phase.

The flow rate in the column may be from around 0.1 mL/min to around 100 mL/min, for example from around 0.5 mL/min to around 50 mL/min, for example from around 0.5 mL/min to around 20 mL/min, for example from around 0.5 mL/min to around 10 mL/min, for example from around 0.5 mL/min to around 5 mL/min. Preferably, the flow rate in the column may be from around 0.5 mL/min to around 5 mL/min.

In a preferred embodiment, the third liquid comprises water. The fourth liquid comprises acetonitrile. The mobile phase initially added to the column most preferably comprises greater than 90% (v/v) of the third liquid and less than 10% (v/v) of the fourth liquid. Preferably the amount (v/v) of the third liquid is abruptly decreased, followed by a plateau. Preferably the amount of the third liquid is decreased to about 50% (v/v) of the mobile phase and the amount of the fourth liquid is increased to about 50% (v/v) of the mobile phase. Preferably the amount of the fourth liquid is abruptly increased, and this increase is simultaneous with the decrease in the amount (v/v) of the third liquid in the mobile phase, followed by a plateau. The step of solid phase enrichment may result in a solid-phase enrichment solution (eluent) having a water content of around 50-60% (w/w). The concentration of oxytocin or oxytocin receptor agonist in the eluent may be around 15 g/L to 25 g/L. Preferably, the flow rate in the column may be from around 0.5 mL/min to around 5 mL/min

The UV absorbance (λ=220 nm) of the mobile phase may be detected with a UV detector and plotted against retention time on the column to produce a solid-phase enrichment chromatogram. The column fractions may be collected and the oxytocin or oxytocin receptor agonist may be separated from impurities on the basis of differing retention times on the solid-phase enrichment chromatogram. The eluent of the major peak may be isolated as a pooled purification fraction from the mobile phase. The pooled purification fraction comprises fractions that are accepted for pooling because they meet the specifications for the final API for purity and impurity limits. Hereinafter, the pooled purification fraction of the mobile phase corresponding to the major peak in the solid-phase enrichment chromatogram (UV absorbance, λ=220 nm) and meeting API specifications is referred to as the “solid-phase enrichment solution.”

Subsequently, analytical liquid chromatography may be performed to assess the purity of the oxytocin or oxytocin receptor agonist in the solid phase enrichment solution. The integrated area under the major peak in the analytical chromatogram (UV absorbance, λ=220 nm) will be proportional to the concentration of the oxytocin or oxytocin receptor agonist, and the integrated area under the minor peaks in the chromatogram will be proportional to the concentrations of impurities, such that a purity of oxytocin or oxytocin receptor agonist may be determined.

Step iv: Distillation to Form Solid Oxytocin, Solid Oxytocin Receptor Agonist or a Solution of Oxytocin or Oxytocin Receptor Agonist Having a Reduced Content of the Solvent of the Oxytocin or Oxytocin Receptor Agonist, (for Example Water) Relative to the Original Solution.

In step iv, the solid-phase enrichment solution referred to above in step iii may be distilled.

Alternatively, in step iv, the liquid chromatography solution referred to above in step ii may be distilled. With reference to steps iv and step v below, the solid-phase enrichment solution or the liquid chromatography solution are referred to as the “original solution.” It should be understood that the terms “reduced content of a solvent of the oxytocin or oxytocin receptor agonist (e.g. water)”/“reduced water content” etc. means that the solvent/water content, which is determined relative to the original solution, may be lower than about 40% w/w, for example, lower than about 30% w/w, for example, lower than about 20% w/w, for example lower than about 15% w/w, for example lower than about 10% w/w, for example from about 5% w/w to about 8% w/w. Water contents within these ranges are preferred as solutions with higher water contents phase separate instead of causing precipitation.

The solvent of the oxytocin or oxytocin receptor agonist may be any solvent in which the oxytocin or oxytocin receptor agonist is soluble in an amount at standard conditions of 0.01 mg/ml or greater, for example 0.05 mg/ml or greater, for example 0.1 mg/ml or greater, for example 0.5 mg/ml or greater, for example 1 mg/ml or greater, for example 5 mg/ml or greater, for example 10 mg/ml or greater, for example 20 mg/ml or greater. The solvent is preferably a hydrophilic solvent. Preferably the (hydrophilic) solvent has a polarity index of greater or equal to 5, greater than equal to 6, greater than or equal to 7, or greater than or equal to 8. The solvent is preferably same as the third liquid as described above with respect to “Step iii)” and is most preferably water.

The distillation may be performed under atmospheric pressure or at a pressure that is lower than atmospheric pressure. The distillation may be performed at a pressure between 0.01 atm and 1 atm, preferably between 0.05 atm and 0.50 atm, most preferably between 0.05 atm and 0.15 atm. The distillation may be performed at a temperature between about 10° C. to about 60° C., most preferably between about 25° C. to about 30° C.

The distillation may be performed on a rotary evaporator or other suitable distillation apparatus.

The final water content by percentage weight may be adjusted downward or upward to be within the preferred ranges, through the addition of either acetonitrile or water, respectively, following the distillation.

The distillation may form solid oxytocin or solid oxytocin receptor agonist, which is hereinafter referred to as the “dry solid.” It is to be understood that dry solid may not be completely dry and may contain some residual solvent or some residual water.

The distillation may form a solution of oxytocin or oxytocin receptor agonist having a reduced content of the solvent of the oxytocin or oxytocin receptor agonist, (for example water) relative to the original solution. The solution with a reduced content of solvent of the oxytocin or oxytocin receptor agonist, (for example water) relative to the original solution is hereinafter referred to as the “distillate solution.”

Oxytocin or oxytocin receptor agonist may be present in the distillate solution comprising oxytocin or oxytocin receptor agonist at a concentration from about 25 g/L to about 45 g/L for precipitation to occur upon combination of the solution with the antisolvent in step vi. Preferably, oxytocin or oxytocin receptor agonist may be present in the distillate solution at a concentration of about 28 g/L to about 40 g/L. Most preferably, oxytocin or oxytocin receptor agonist may be present in the distillate solution at a concentration of about 35 g/L.

A solution of acetonitrile and water, with a reduced water content of about 13% w/w or lower is a preferred distillate solution as this results in greater consistency in the purity of product oxytocin or product oxytocin receptor agonist between batches and a greater ability for the product oxytocin or product oxytocin receptor agonist to be analysed for residual solvent via gas chromatography after the product oxytocin or oxytocin receptor agonist is precipitated in step vi. Most preferably, the distillate solution comprising oxytocin or oxytocin receptor agonist comprises acetonitrile and water, with a reduced water content of from about 5% w/w to about 8% w/w.

Step v: Where the Distillation Process in Step iv Results in the Formation of Dry Solid Oxytocin or Dry Solid Oxytocin Receptor Agonist, Re-Dissolving the Solid Oxytocin or Solid Oxytocin Receptor Agonist to Form a Solution Comprising Oxytocin or a Oxytocin Receptor Agonist Having a Reduced Content of a Solvent of the Oxytocin or Oxytocin Receptor Agonist (e.g. Water) Relative to the Original Solution

If present, the dry solid oxytocin or oxytocin receptor agonist formed by step iv) may be re-dissolved in a solvent of the oxytocin or oxytocin receptor agonist (as described above), which is preferably the same as the third liquid as described above with respect to step iii), and a hydrophobic liquid (which is preferably the same as the fourth liquid as described above with respect to step iii) to form a solution having a sufficiently reduced content of the solvent of the oxytocin or oxytocin receptor agonist relative to the original solution such that the addition of the antisolvent in step vi) causes precipitation, rather than phase separation. The solution formed in this step, which has a reduced content of solvent of the oxytocin or oxytocin receptor agonist, (for example water) relative to the original solution, is hereinafter referred to as the “concentrated solution.”

The solvent of the oxytocin or oxytocin receptor agonist and the “reduced content of a solvent of the oxytocin or oxytocin receptor agonist (e.g. water)”/“reduced water content” etc. may all be as described above with respect to step iv).

Oxytocin or oxytocin receptor agonist may be present in the concentrated solution comprising oxytocin or oxytocin receptor agonist at a concentration from about 25 g/L to about 45 g/L for precipitation to occur upon combination of the solution with the antisolvent in step vi. Preferably, oxytocin or oxytocin receptor agonist may be present in the concentrated solution at a concentration of about 28 g/L to about 40 g/L. Most preferably, oxytocin or oxytocin receptor agonist may be present in the concentrated solution at a concentration of about 35 g/L.

A solution of acetonitrile and water, with a reduced water content of about 13% w/w or lower is a preferred concentrated solution as this results in greater consistency in the purity of product oxytocin or product oxytocin receptor agonist between batches and a greater ability for the product oxytocin or product oxytocin receptor agonist to be analysed for residual solvent via gas chromatography after the product oxytocin or oxytocin receptor agonist is precipitated in step vi. Most preferably, the concentrated solution comprising oxytocin or oxytocin receptor agonist comprises acetonitrile and water, with a reduced water content of from about 5% w/w to about 8% w/w.

Step vi: Combining an Antisolvent with a Solution Comprising Oxytocin or Oxytocin Receptor Agonist Having a Reduced Content of a Solvent of the Oxytocin or Oxytocin Receptor Agonist (e.g. Water) (for Example the Solution Formed by Step iv) or Step v)) so as to Precipitate a Product Oxytocin or a Product Oxytocin Receptor Agonist from the Solution.

The solution comprising oxytocin or oxytocin receptor agonist in step vi may be the distillate solution of step iv or the concentrated solution of step v.

As described above with respect to the method overall, the antisolvent may be a non-polar liquid, for example a liquid having a polarity index of less than or equal to 8, for example less than or equal to 7, for example less than or equal to 6, for example less than or equal to 5, for example less than or equal to 4. Preferably, the antisolvent has a polarity index greater than or equal to 2 and less than or equal to 5. The antisolvent may be an aprotic non-polar liquid, for example an ester or an ether, for example ethyl acetate (polarity index=4.4), diethyl ether (polarity index=2.8), ethyl tert-butyl ether, di-isopropyl ether (polarity index=2.2), 2-methyltetrahydrofuran, propyl acetate, isopropyl acetate, methyl propionate, ethyl propionate or methyl tert-butyl ether (polarity index=2.5). Methyl tert-butyl ether is the preferred antisolvent. Thus, in one embodiment, the antisolvent in step iv) is methyl tert-butyl ether.

As described above with respect to the method overall, oxytocin or oxytocin receptor agonist may be present in the solution comprising oxytocin or oxytocin receptor agonist at a concentration from about 25 g/L to about 45 g/L for precipitation to occur upon combination of the solution with the antisolvent in step vi. Preferably, oxytocin or oxytocin receptor agonist may be present in the solution at a concentration of about 28 g/L to about 40 g/L. Most preferably, oxytocin or oxytocin receptor agonist may be present in the solution at a concentration of about 35 g/L. As described above with respect to the method overall, a solution of acetonitrile and water, with a reduced water content of about 13% w/w or lower is a preferred solution as this results in greater consistency in the purity of product oxytocin or product oxytocin receptor agonist between batches and a greater ability for the product oxytocin or product oxytocin receptor agonist to be analysed for residual solvent via gas chromatography after the product oxytocin or oxytocin receptor agonist is precipitated in step vi. Most preferably, the solution comprising oxytocin or oxytocin receptor agonist comprises acetonitrile and water, with a reduced water content of from about 5% w/w to about 8% w/w.

Instead of adding antisolvent to the solution, preferably the solution comprising oxytocin or oxytocin agonist may be added to the antisolvent, as this minimises the loss of product.

It was found that adding the solution comprising oxytocin or oxytocin receptor agonist to the antisolvent quickly, for example over a period of 20 minutes or less, for example over a period of 15 minutes or less, for example over a period of 10 minutes or less, for example over a period of 7.5 minutes or less, for example over a period of 5 minutes or less results in the formation of smaller particles, which in turn results in a greater consistency in the purity of product oxytocin or product oxytocin receptor agonist between batches and greater ability for the product oxytocin or oxytocin receptor agonist to be analysed for residual solvent via gas chromatography. Most preferred was addition over a period of 5 minutes or less.

Preferably, the solution comprising oxytocin or oxytocin receptor agonist may be added to 2 to 20 volume equivalents of the antisolvent, for instance 5 volume equivalents of antisolvent.

Prior to the addition of solution the antisolvent, the antisolvent may be at a temperature lower than ambient temperature, for example at a temperature of from −40° C. to 10° C., preferably at a temperature of from −20° C. to 0° C.

The precipitated product may be agitated, such as by stirring, while aging for several hours, for instance 3 hours. Preferably, the precipitated product may be aged at a temperature of from 0° C. to 60° C., most preferably at a temperature of from 15° C. to 30° C.

Following the precipitation of the product oxytocin or a product oxytocin receptor agonist from the solution, the product oxytocin or a product oxytocin receptor agonist may be vacuum dried for a period of from 1 hour to 1 week, for example from 2 hours to 6 days, for example from 4 hours to 5 days, for example from 6 hours to 4 days, for example from 12 hours to 4 hours, for example from 1 day to 3 days. The temperature at which the drying takes place is preferably from about −196° C. to about 100° C., for example from about −50° C. to about 80° C., for example from about −20° C. to about 60° C., for example from about −5° C. to about 45° C., for example from about 0° C. to about 40° C., for example from about 5° C. to about 35° C., for example from about 10° C. to about 30° C., for example from about 16° C. to about 28° C., for example about 22° C. Following the drying, the product oxytocin or a product oxytocin receptor agonist may be placed in nitrogen purged Nalgene bottles and stored at a temperature of from −196° C. to about 50° C., for example from about −50° C. to about 40° C., for example from about −20° C. to about 35° C., for example from about −5° C. to about 25° C., for example from about 0° C. to about 20° C., for example from about 0° C. to about 10° C., for example from about 2° C. to about 8° C., for example about 5° C.

The oxytocin receptor agonist may be a selective oxytocin receptor agonist. The term “selective oxytocin receptor agonist” may refer to a compound that has a greater agonist activity at a human oxytocin (hOT) receptor than at a human vasopressin receptor (e.g., a human V2 (hV2) receptor) and has a greater selectivity compared to oxytocin. Agonist activity may be expressed as EC₅₀ (half-maximal effective concentration), and selectivity may be expressed as the ratio of EC₅₀ values (i.e., EC₅₀ hv2/EC₅₀ hOT), both as described under the heading “In vitro receptor assays” in WO2009/122285 (International Patent Application No. PCT/IB2009/005351) of Ferring B.V.). Oxytocin has an hV2/hOT selectivity ratio of 3; and carbetocin has a hV2/EC₅₀ selectivity ratio of 244 (see Table 2 in WO2009/122285). The oxytocin receptor agonist may have an hV2/hOT selectivity ratio of at least 50 (e.g., at least 100, at least 200, at least 244, at least 300, at least 500, at least 1,000, at least 2,000, at least 3,000, at least 5,000, at least 10,000, at least 20,000, or at least 30,000).

The oxytocin or oxytocin receptor agonist may be a compound of formula (I):

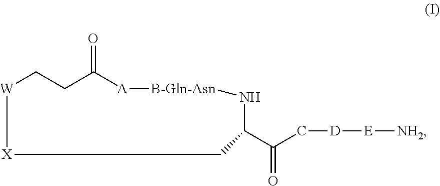

in which each of W and X, independently, is CH₂ and S, provided that W and X are not both CH₂; A is an amino acid selected from the group consisting of alanine substituted on the side chain with a 5- or 6-membered heteroaromatic ring; tyrosine; and phenylalanine substituted on the phenyl ring with halogen, C₁₋₄ alkoxy, C₁₋₄ alkylhydroxy, C₁₋₄ alkyl or amino; B is an amino acid selected from the group consisting of isoleucine; and glycine substituted on the α-carbon with C₄₋₆ cycloalkyl; C is an amino acid selected from the group consisting of proline, optionally substituted on the side chain (for example at the 4-position) with hydroxyl, C₁₋₄ alkoxy, halogen or azide, and proline having its side chain optionally interrupted by a heteroatom and which optionally interrupted side chain is optionally substituted with C₁₋₄ alkyl (for example, at the 4-position); D is an amino acid selected from the group consisting of leucine; homoleucine; isoleucine; and glycine substituted on the α-carbon with C₄₋₆ cycloalkyl; and E is an amino acid selected from the group consisting of glycine and azaglycine. Alternatively, the oxytocin or an oxytocin receptor agonist may be a compound of formula (II):

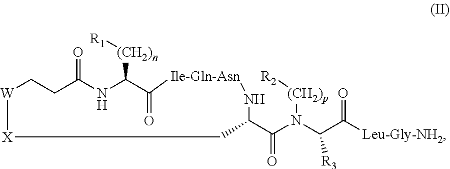

in which n is 0, 1 or 2; p is 0, 1, 2, 3, 4, 5 or 6; R₁ is aryl optionally substituted with at least one OH, F, Cl, Br, alkyl, or O-alkyl substituent; R₂ is R₄, H, alkyl, cycloalkyl, aryl, a 5-membered heteroaromatic ring system, or a 6-membered heteroaromatic ring system; R₃ is H or a covalent bond to R₂, when R₂ is R₄, to form a ring structure; R₄ is a C₁₋₆ alkylene moiety substituted with at least one O-alkyl, S-alkyl or OH substituent; each of W and X, independently, is CH₂ and S, provided that W and X are not both CH₂; alkyl is C₁₋₆ straight or C₄₋₈ branched chain alkyl and optionally has at least one hydroxyl substituent; aryl is unsubstituted or substituted phenyl; and cycloalkyl is C₃₋₆ cycloalkyl and optionally has at least one hydroxyl substituent;
when R₂ in formula (II) is H, p is 1, R₃ is H, n is 1 and W and X are both S, R₁ may not be 4-hydroxyphenyl, when R₂ is H, p is 0, R₃ is H, n is 1 and W and X are both S, R₁ may not be 4-hydroxyphenyl, a compound of formula (II) may not be [I-β-Mpa,7-Sar]OT or deamino[7-glycine] oxytocin;
the groups in formula (II) may optionally comprise substituent moieties selected from fluorine (F), chlorine (Cl) and bromine (Br) atoms and alkyl, hydroxy (OH), alkoxy (O-alkyl) and alkylthio (S-alkyl).

Alternatively, the oxytocin or a oxytocin receptor agonist may be carbetocin [also known as (1-desamino-1-monocarba-2(O-methyl)-tyrosine)oxytocin and 1-butanoic acid-2-(O-methy-L-tyrosine)-1-carbaoxytocin].

Other exemplary compounds of formulae (I) and (II) have been described, for example in WO2011/035330 and WO2009/122285, which are herein incorporated by reference. According to the invention in a further aspect there is provided a pharmaceutical composition including oxytocin or oxytocin receptor agonist made according to a method of the invention. The pharmaceutical composition of the invention may be for use as a medicament. The pharmaceutical composition of the invention may be for use in the treatment of a neurological disorder or reproductive disorder, for example for use in the treatment of Prader-Willi syndrome, for example for use in the treatment or prevention of uterine atony, for example following vaginal delivery of the infant, delivery of the infant by Caesarean section, or for use in the treatment or prevention of uterine atony in a patient who is at risk of developing postpartum haemorrhage (PPH); and/or for use in the treatment or prevention of excessive bleeding following vaginal delivery.

The invention may further be defined in the following clauses:
1. A method of manufacturing oxytocin or oxytocin receptor agonist comprising a step of combining an antisolvent with a solution comprising oxytocin or oxytocin receptor agonist so as to precipitate a product oxytocin or a product oxytocin receptor agonist from the solution.
2. A method of manufacturing oxytocin or oxytocin receptor agonist comprising the steps of:
   i) Optionally synthesising or providing crude oxytocin or crude oxytocin receptor agonist (for example, a solution comprising crude oxytocin or a solution comprising crude oxytocin receptor agonist);
   ii) Optionally performing liquid chromatography, for example HPLC, for example reverse phase HPLC on crude oxytocin or a crude oxytocin receptor agonist (for example, on the (e.g. solution of) crude oxytocin or crude oxytocin receptor agonist formed in step i);
   iii) Optionally performing a step of solid-phase enrichment (for example on the (e.g. solution of) oxytocin or oxytocin receptor agonist formed in step i), or step ii)) to provide a solution comprising oxytocin or a oxytocin receptor agonist;
   iv) Optionally distilling a solution comprising oxytocin or a oxytocin receptor agonist (for example, the solution formed in step ii) or step iii)) to form solid crude oxytocin or a solid crude oxytocin receptor agonist, or a solution of oxytocin or oxytocin receptor agonist having a reduced content of a solvent of the oxytocin or oxytocin receptor agonist (e.g. water);
   v) optionally redissolving the solid oxytocin or solid oxytocin receptor agonist to form a solution comprising oxytocin or a oxytocin receptor agonist having a reduced content of a solvent of the oxytocin or oxytocin receptor agonist (e.g. water), if solid oxytocin or solid oxytocin receptor agonist is present following step iv); and
   vi) combining an antisolvent with a solution comprising oxytocin or oxytocin receptor agonist (for example a solution having a reduced content of a solvent of the oxytocin or oxytocin receptor agonist (e.g. water), such as the solution formed by step iv or step v)) so as to precipitate a product oxytocin or a product oxytocin receptor agonist from the solution.
3. A method of manufacturing oxytocin or oxytocin receptor agonist comprising the steps of:
   i) synthesising or providing crude oxytocin or crude oxytocin receptor agonist (for example, a solution comprising crude oxytocin or a solution comprising crude oxytocin receptor agonist);
   ii) performing liquid chromatography, for example HPLC, for example reverse phase HPLC, on the (e.g. solution of) crude oxytocin or crude oxytocin receptor agonist formed in step i);
   iii) performing a step of solid-phase enrichment on the (e.g. solution of) oxytocin or oxytocin receptor agonist formed in step ii) to provide a solution comprising oxytocin or a oxytocin receptor agonist;
   iv) distilling the solution formed in step iii) to form solid oxytocin or a solid oxytocin receptor agonist or a solution of oxytocin or oxytocin receptor agonist having a reduced content of a solvent of the oxytocin or oxytocin receptor agonist (e.g. water);
   v) redissolving the solid oxytocin or solid oxytocin receptor agonist to form a solution comprising oxytocin or a oxytocin receptor agonist having a reduced content of a solvent of the oxytocin or oxytocin receptor agonist (e.g. water) if solid oxytocin or solid oxytocin receptor agonist is present following step iv); and
   vi) combining an antisolvent with the solution comprising oxytocin or oxytocin receptor agonist having a reduced content of a solvent of the oxytocin or oxytocin receptor agonist (e.g. water) formed by step iv) or step v) so as to precipitate a product oxytocin or a product oxytocin receptor agonist from the solution.
4. A method according to any preceding clause wherein the crude oxytocin or crude oxytocin receptor agonist has a purity of less than 97%, for example less than 96%, for example less than 95%, for example less than 92.5%, for example less than 90%.
5. A method according to any preceding clause wherein the (product) oxytocin or oxytocin receptor agonist (that is, the product of the step of combining the solution with antisolvent) has a purity greater than or equal to 97.5%, for example greater than or equal to 98%, for example greater than or equal to 98.5%, for example greater than or equal to 99%, for example greater than or equal to 99.25%, for example greater than or equal to 99.5%.
6. A method according to any preceding clause wherein a solvent in any solution comprising oxytocin or oxytocin receptor agonist, for example the carbetocin solution which is combined with the antisolvent, is a polar solvent, for example a solvent having a polarity index of at least 5, for example a mixture of a polar aprotic compound having a polarity index of at least 5 and a polar protic compound having a polarity index of at least 5.
7. A method according to clause 6 wherein the polar solvent comprises a mixture of water and acetonitrile, for example a mixture comprising 90% acetonitrile 10% water (v/v).
8. A method according to any preceding clause wherein the antisolvent has a polarity index of less than or equal to 8, for example a polarity index of less than or equal to 6, for example an ester or an ether, for example ethyl acetate or methyl tert-butyl ether.
9. A method according to any of clauses 2 to 8 wherein the step of performing liquid chromatography comprises the use of a column together with a mobile phase comprising a first liquid and/or a second liquid, for example wherein the first liquid comprises an ammonium salt solution and/or the second liquid comprises methanol.
10. A method according to clause 9 wherein the ammonium salt solution is an ammonium acetate solution, for example an aqueous ammonium acetate solution.
11. A method according to clause 9 or clause 10 wherein the ammonium salt solution comprises 1 mM/L to 10 M/L ammonium salt, for example from 2 mM/L to 5 M/L ammonium salt, for example from 5 mM/L to 2M/L ammonium salt, for example from 10 mM/L to 1 M/L ammonium salt, for example from 20 mM/L to 500 mM/L ammonium salt, for example from 50 mM/L to 200 mM/L ammonium salt, for example about 100 mM/L ammonium salt.
12. A method according to any of clauses 8 to 11 wherein the second liquid comprises 50% or more methanol, for example 60% or more methanol, for example 70% or more methanol, for example 80% or more methanol, for example 90% or more methanol, for example 95% or more methanol, for example 98% or more methanol, for example 99% or more methanol, for example substantially pure methanol, for example pure methanol.
13. A method according to any of clauses 2 to 12 wherein the step of solid phase enrichment (step iii) comprises the use of a third liquid and a fourth liquid.
14. A method according to clause 13 wherein the third liquid comprises water and/or the fourth liquid comprises acetonitrile.
15. A method according to any preceding clause wherein the oxytocin or selective oxytocin receptor agonist is a compound of formula (I):

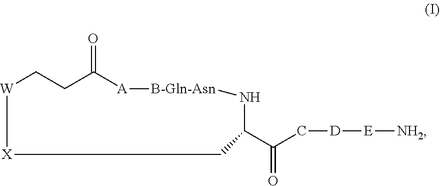

in which each of W and X, independently, is CH₂ and S, provided that W and X are not both CH₂; A is an amino selected from the group consisting of alanine substituted on the side chain with a 5- or 6-membered heteroaromatic ring; tyrosine; and phenylalanine substituted on the phenyl ring with halogen, C₁₋₄ alkoxy, C₁₋₄ alkylhydroxy, C₁₋₄ alkyl or amino; B is an amino acid selected from the group consisting of isoleucine; and glycine substituted on the α-carbon with C₄₋₆ cycloalkyl; C is an amino acid selected from the group consisting of proline, optionally substituted on the side chain (for example at the 4-position) with hydroxyl, C₁₋₄ alkoxy, halogen or azide, and proline having its side chain optionally interrupted by a heteroatom and which optionally interrupted side chain is optionally substituted with 01-4 alkyl (for example, at the 4-position); D is an amino acid selected from the group consisting of leucine; homoleucine; isoleucine; and glycine substituted on the α-carbon with C₄₋₆ cycloalkyl; and E is an amino acid selected from the group consisting of glycine and azaglycine; or
wherein the oxytocin or a selective oxytocin receptor agonist is a compound of formula (II):

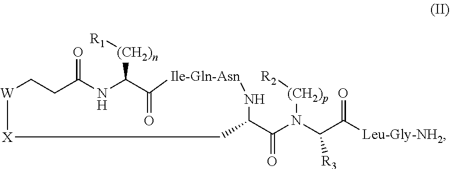

in which n is 0, 1 or 2; p is 0, 1, 2, 3, 4, 5 or 6; R₁ is aryl optionally substituted with at least one OH, F, Cl, Br, alkyl, or O-alkyl substituent; R₂ is R₄, H, alkyl, cycloalkyl, aryl, a 5-membered heteroaromatic ring system, or a 6-membered heteroaromatic ring system; R₃ is H or a covalent bond to R₂, when R₂ is R₄, to form a ring structure; R₄ is a C₁₋₆ alkylene moiety substituted with at least one O-alkyl, S-alkyl or OH substituent; each of W and X, independently, is CH₂ and S, provided that W and X are not both CH₂; alkyl is C₁₋₆ straight or C₄₋₈ branched chain alkyl and optionally has at least one hydroxyl substituent; aryl is unsubstituted or substituted phenyl; and cycloalkyl is C₃₋₆ cycloalkyl and optionally has at least one hydroxyl substituent;
when R₂ in formula (II) is H, p is 1, R₃ is H, n is 1 and W and X are both S, R₁ may not be 4-hydroxyphenyl, when R₂ is H, p is 0, R₃ is H, n is 1 and W and X are both S, R₁ may not be 4-hydroxyphenyl, a compound of formula (II) may not be [I-β-Mpa,7-Sar]OT or deamino[7-glycine] oxytocin;
the groups in formula (II) may optionally comprise substituent moieties selected from fluorine (F), chlorine (Cl) and bromine (Br) atoms and alkyl, hydroxy (OH), alkoxy (O-alkyl) and alkylthio (S-alkyl); or
wherein the oxytocin or a selective oxytocin receptor agonist is carbetocin.
16. A pharmaceutical composition including an oxytocin or oxytocin receptor agonist made according to the method of any preceding clause.
17. The pharmaceutical composition of clause 16 for use as a medicament.
18. The pharmaceutical composition of clause 16 for use in the treatment of a neurological disorder or reproductive disorder, for example for use in the treatment of Prader-Willi syndrome, for example for use in the treatment or prevention of uterine atony, for example following vaginal delivery of the infant, delivery of the infant by Caesarean section, or for use in the treatment or prevention of uterine atony in a patient who is at risk of developing PPH; and/or for use in the treatment or prevention of excessive bleeding following vaginal delivery.

The invention is further disclosed in the following examples which are not to be construed as limiting, but merely as examples. In some cases, the examples are considered to be preferred embodiments of the present invention.

### EXAMPLES

### Example 1—Method of Producing a Pure Product, e.g. Pure Carbetocin

### Step i: Synthesis

Crude carbetocin of purity approx. 91% was obtained by synthetic methods analogous to those described in WO2009/122285 (International Patent Application No. PCT/IB2009/005351) of Ferring B.V.

### Step ii: Liquid Chromatography

Purification of the crude carbetocin was carried out by RP-HPLC using a 1 cm Phenomenex Luna C18(2) 20 mm×250 mm column, with pore size 100 Å. Alternatively, a 1 cm×250 mm Kromasil C-18 100-10 column could be used. The wavelength was 220 mm.

250 mg of the crude carbetocin was dissolved in 95/5% H₂O/MeOH. The solution was stirred until the carbetocin was dissolved, and the solution then loaded onto the column.

The gradient used is shown in table 1 below. The first liquid (described in table 1 as Mobile Phase A) consisted of a solution consisting of a 200 mM aqueous solution of ammonium acetate. The second liquid (described in table 1 as Mobile Phase B) was pure methanol.

TABLE 1
RP-HPLC Gradient

| | % Mobile | % Mobile | Flow |
|---|---|---|---|
| Time | Phase A | Phase B | (mL/min) |
| 0.0 | 95.0 | 5.0 | 3.8 |
| 5.0 | 52.0 | 48.0 | 3.8 |
| 37.0 | 44.0 | 56.0 | 3.8 |
| 39.0 | 5.0 | 95.0 | 3.8 |
| 41.0 | 5.0 | 95.0 | 3.8 |
| 41.1 | 95.0 | 5.0 | 3.8 |
| 45.0 | 95.0 | 5.0 | 3.8 |

Step ii) formed a MeOH/H₂O solution comprising carbetocin (liquid chromatography solution).

The purity of the carbetocin in the liquid chromatography solution following the purification step was 97.9% as measured by analytical HPLC. The single largest impurity was measured at 0.44%, with a relative retention time (RRT) of 0.16. The analytical HPLC was carried out on a Zorbax SB-phenyl column, 75×4.6 mm, 3.5 μm. Mobile phase A consisted of 1900 mL H2O, 100 mL triethylammonium phosphate and 4 mL phosphoric acid. Mobile phase B consisted of acetonitrile. The wavelength was 220 mm.

The gradient used for the analytical HPLC is shown in Table 2 below.

TABLE 2
Analytical HPLC Gradient

| Time | Mobile | Mobile | Flow Rate |
|---|---|---|---|
| (min) | Phase A (%) | Phase B (%) | (mL/min) |
| 0 | 80.0 | 20.0 | 2.0 |
| 15 | 60.0 | 40.0 | 2.0 |
| 17 | 5.0 | 95.0 | 2.0 |
| 17.2 | 20.0 | 20.0 | 2.0 |
| 22.0 | 20.0 | 20.0 | 2.0 |

### Step iii: Solid Phase Enrichment

The carbetocin MeOH/H₂O solution prepared by Step ii underwent concentration by a solid phase enrichment step to form a MeCN/H₂O solution.

The solid phase enrichment step comprised diluting the pooled purification fraction (liquid chromatography solution) obtained from step ii: (1 vol. equiv.) with deionized water (2 vol. equiv.)

The solution was then loaded to an Amberchrom™ CG300M column and flushed with the water to remove methanol and ammonium acetate. The solution was then eluted with a 50/50 mixture of water and MeCN. The gradient is shown in Table 3 below.

TABLE 3
Solid Phase Enrichment Gradient

| | % Mobile | % Mobile | Flow |
|---|---|---|---|
| Time | Phase A | Phase B | mL/min |
| 0 | 100.0 | 0.0 | 0.7 |
| 10 | 100.0 | 0.0 | 0.7 |
| 10.1 | 50.0 | 50.0 | 0.7 |
| 400 | 50.0 | 50.0 | 0.7 |

The wavelength used in the solid phase enrichment step was 220 nm.

The amount of water present in the solid-phase enrichment solution following solid phase enrichment was found to be around 40% to 60% w/w. The amount of carbetocin in the solid-phase enrichment solution was found to be approximately 20 g/L as measured by analytical HPLC. The analytical HPLC was carried out as described above with respect to Example 1, step ii.

### Steps iv and vi: Distillation and Precipitation (Step v not Carried Out),

The MeCN/H₂O carbetocin solution formed by Step iii underwent the following steps:
a) Distillation to form a solution having a reduced water content (from 40% to 60% w/w water to from 5% to 8% w/w water). This increased the carbetocin concentration from approximately 20 g/L to approximately 35 g/L. (step iv)
b) The H₂O/MeCN carbetocin solution was then added to 5 vol. equiv. 0° C. methyl tert-butyl ether antisolvent over a period of 4 minutes to precipitate pure carbetocin of purity 99.2% as measured by analytical HPLC and yield 86.3% (step vi). Following drying of the precipitate, the sample was humidified prior to testing for residual solvent. The humidification consisted of passing 22° C. humidified N₂ at a 4 standard cubic feet per hour flow across pure carbetocin in an unjacketed filter. Humidification was run from 5 h to 18 h on different samples. The relative humidity at the completion of the humidification ranged from 30 to 40%. The amount of residual methyl tert-butyl ether was calculated at 1780 ppm (below the pharmaceutically acceptability threshold of 5000 ppm). The residual solvent analysis was performed by gas chromatography, with parameters as in Table 4 below. No residual MeCN was detected.

TABLE 4
Gas chromatography parameters

| Column: | Supelcocowax ™-10, | |
|---|---|---|
| | 30 m × 0.53 mm, 2 μm film | |
| Oven Temperature: | Initial: 35° C. (hold 2 min) | |
| | Ramp: 3° C./min to 70° C. (hold 0 min) | |
| | Ramp: 5° C./min to 200° C. (hold 0 min) | |
| Injector Temperature | 180° C. | |
| Flame Ionisation | 200° C. | |
| Detector Temperature | | |
| Pressure | Approximately 5.5 psi He at 35° C. | |
| Column flow (constant) | Approximately 8 mL/min | |
| Inlet | Splitless | |
| Injection volume | 0.5 | μL |
| Run time | 39.67 | min |

The method provides high purity carbetocin in an acceptable yield without the need for a lyophilisation step. The removal of the lyophilisation “bottleneck” means that large volumes of carbetocin can be purified at a reasonable cost. Accordingly, this method is suitable for producing the relatively large amounts of peptide required in the treatment of Prader-Willi Syndrome, for example.

### Example 2—Method of Producing a Pure Product, e.g. Pure Carbetocin

### Step i: Synthesis

Crude carbetocin of purity approx. 91% was obtained by synthetic methods analogous to those described in WO2009/122285 (International Patent Application No. PCT/162009/005351) of Ferring B.V.

### Step ii: Liquid Chromatography

Purification of the crude carbetocin was carried out by RP-HPLC using a 1 cm Phenomenex™ Luna C18(2) 20 mm×250 mm column, with pore size 100 Å. Alternatively, a 1 cm×250 mm Kromasil™ C-18 100-10 column could be used. The wavelength was 220 mm.

250 mg of the crude carbetocin was dissolved in 95/5% H₂O/MeOH. The solution was stirred until the carbetocin was dissolved, and the solution then loaded onto the column.

The gradient used is shown in Table 5 below. The first liquid (described in Table 5 as Mobile Phase A) consisted of a solution consisting of a 200 mM aqueous solution of ammonium acetate. The second liquid (described in Table 5 as Mobile Phase B) was pure methanol.

TABLE 5
RP-HPLC Gradient

| | % Mobile | % Mobile | Flow |
|---|---|---|---|
| Time | Phase A | Phase B | (mL/min) |
| 0.0 | 95.0 | 5.0 | 3.8 |
| 5.0 | 52.0 | 48.0 | 3.8 |
| 37.0 | 44.0 | 56.0 | 3.8 |
| 39.0 | 5.0 | 95.0 | 3.8 |
| 41.0 | 5.0 | 95.0 | 3.8 |
| 41.1 | 95.0 | 5.0 | 3.8 |
| 45.0 | 95.0 | 5.0 | 3.8 |

Step ii) formed a MeOH/H₂O solution comprising carbetocin (liquid chromatography solution).

The purity following the purification step was 97.9%. The single largest impurity was measured at 0.44%, with a relative retention time (RRT) of 0.16. The analytical HPLC was carried out as described above in Example 1, step ii.

### Step iii: Solid Phase Enrichment

The carbetocin MeOH/H₂O solution prepared by Step ii underwent concentration by a solid phase enrichment step to form an MeCN/H₂O solution.

The solid phase enrichment step comprised diluting the pooled purification fraction (liquid chromatography solution) obtained from step ii: (1 vol. equiv.) with deionized water (2 vol. equiv.)

The solution was then loaded to an Amberchrom™ CG300M column and flushed with the water to remove methanol and ammonium acetate. The solution was then eluted with a 50/50 mixture of water and MeCN. The gradient is shown in Table 6 below.

TABLE 6
Solid Phase Enrichment Gradient

| | % Mobile | % Mobile | Flow |
|---|---|---|---|
| Time | Phase A | Phase B | mL/min |
| 0 | 100.0 | 0.0 | 0.7 |
| 10 | 100.0 | 0.0 | 0.7 |
| 10.1 | 50.0 | 50.0 | 0.7 |
| 400 | 50.0 | 50.0 | 0.7 |

The wavelength used in the solid phase enrichment step was 220 nm.

The amount of water present in the solid-phase enrichment solution following solid phase enrichment was found to be around 40% to 60% w/w. The amount of carbetocin in the solid-phase enrichment solution was found to be approximately 20 g/L as measured by analytical HPLC. The analytical HPLC was carried out as described above with respect to Example 1, step ii.

### Steps iv, v and vi: Distillation, Re-Dissolution and Precipitation

The MeCN/H₂O carbetocin solution formed by Step iii underwent the following steps:
a) Distillation to form solid carbetocin (step iv)
b) Re-dissolving the solid carbetocin in MeCN and H₂O to form a solution having a H₂O content of 5% to 8% w/w (step v).
c) The H₂O/MeCN carbetocin solution was then added to 5 vol. equiv. 0° C. methyl tert-butyl ether antisolvent over a period of 4 minutes to precipitate pure carbetocin of purity 99.2% as measured by analytical HPLC and yield 86.3% (step vi). Following drying of the precipitate, the sample was humidified prior to testing for residual solvent. The humidification consisted of passing 22° C. humidified N₂ at a 4 standard cubic feet per hour flow across pure carbetocin in an unjacketed filter. Humidification was run from 5 h to 18 h on different samples. The relative humidity at the completion of the humidification ranged from 30 to 40%. The amount of residual methyl tert-butyl ether was calculated at 1780 ppm (below the pharmaceutically acceptability threshold of 5000 ppm). The residual solvent analysis was performed by gas chromatography, with parameters as in Example 1, step vi above. No residual MeCN was detected.
   The method provides high purity carbetocin in an acceptable yield without the need for a lyophilisation step. The removal of the lyophilisation “bottleneck” means that large volumes of carbetocin can be purified at a reasonable cost. Accordingly, this method is suitable for producing the relatively large amounts of peptide required in the treatment of Prader-Willi Syndrome, for example.

1-31. (canceled)

32. A method of manufacturing oxytocin or oxytocin receptor agonist comprising:
(i) performing liquid chromatography on a crude oxytocin or crude oxytocin receptor to obtain a liquid chromatography solution comprising oxytocin or oxytocin receptor agonist;
(ii) performing solid-phase enrichment on the liquid chromatography solution obtained in step (i) to obtain a solid-phase enrichment solution comprising oxytocin or a oxytocin receptor agonist;
(iii) distilling the solid-phase enrichment solution obtained in step (ii) to obtain dry solid oxytocin or dry solid oxytocin receptor agonist or a distillate solution of oxytocin or oxytocin receptor agonist having a reduced content of a solvent of the oxytocin or oxytocin receptor agonist relative to the solid-phase enrichment solution;
(iv) if present following step (iii) redissolving the dry solid oxytocin or dry solid oxytocin receptor agonist to obtain a concentrated solution comprising oxytocin or oxytocin receptor agonist having a reduced content of a solvent of the oxytocin or oxytocin receptor agonist relative to the solid-phase enrichment solution; and
(v) combining an antisolvent with the distillate of step (iii) or concentrated solution of step (iv) to precipitate a product oxytocin or a product oxytocin receptor agonist.

33. A method according to claim 32, wherein the crude oxytocin or crude oxytocin receptor agonist has a purity of less than 97%

34. A method according to claim 32, wherein the product oxytocin or product oxytocin receptor agonist has a purity greater than or equal to 97.5%.

35. A method according to claim 32, wherein a solvent in any solution comprising oxytocin or oxytocin receptor agonist is a polar solvent having a polarity index of at least 5.

36. A method according to claim 35, wherein the polar solvent comprises a mixture of water and acetonitrile.

37. A method according to claim 36, wherein the polar solvent comprises a mixture comprising 90% acetonitrile 10% water (v/v).

38. A method according to claim 36, wherein the polar solvent comprises a mixture of water and acetonitrile with a water content of from about 5% w/w to about 8% w/w.

39. A method according to claim 32, wherein oxytocin or oxytocin receptor agonist is present at a concentration of from about 25 g/L to about 45 g/L in the distillate of step (iii) or the concentrated solution of step (iv).

40. A method according to claim 32, wherein the oxytocin or oxytocin receptor agonist is carbetocin.

41. A method according to claim 32, wherein the antisolvent is an ester or an ether.

42. A method according to claim 32, wherein the antisolvent is selected from ethyl acetate and methyl tert-butyl ether.

43. A method according to claim 32, wherein liquid chromatography is performed with a column and a mobile phase comprising a first liquid and a second liquid, wherein the first liquid comprises an ammonium salt solution and the second liquid comprises methanol.

44. A method according to claim 43, wherein the ammonium salt solution is an aqueous ammonium acetate solution.

45. A method according to claim 44, wherein the ammonium salt solution comprises from 1 mM to 10 M ammonium salt.

46. A method according to claim 43, wherein the second liquid comprises 50% v/v or more methanol.

47. A method according to claim 32, wherein the solid phase enrichment is performed with a third liquid comprising water and a fourth liquid comprising acetonitrile.

48. A method according to claim 32, wherein the oxytocin or oxytocin receptor agonist is
(a) a compound of formula (I): 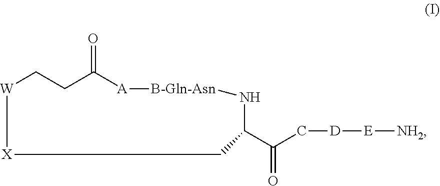
in which:
each of W and X independently is CH₂ or S, provided that W and X are not both CH₂;
A is an amino selected from alanine substituted on its side chain with a 5- or 6-membered heteroaromatic ring; tyrosine; and phenylalanine substituted on its phenyl ring with halogen, C1-C4 alkoxy, C1-C4 alkylhydroxy, C1-C4 alkyl, or amino;
B is an amino acid selected from isoleucine and glycine substituted on its α-carbon with C4-C6 cycloalkyl;
C is an amino acid selected from proline optionally substituted on its side chain with hydroxyl, C1-C4 alkoxy, halogen, or azide, and proline having its side chain optionally interrupted by a heteroatom wherein the optionally interrupted side chain is optionally substituted with C1-C4 alkyl;
D is an amino acid selected from leucine; homoleucine; isoleucine; and glycine substituted on its α-carbon with C4-C6 cycloalkyl; and
E is an amino acid selected from glycine and azaglycine; or
(b) a compound of formula (II): 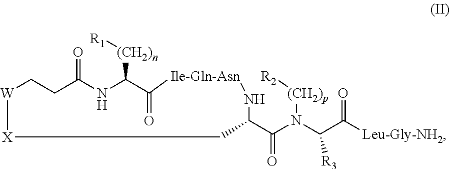
in which:
n is 0, 1 or 2;
p is 0, 1, 2, 3, 4, 5 or 6;
R₁ is Aryl optionally substituted with at least one OH, F, Cl, Br, Alkyl, or O-Alkyl substituent;
R₂ is selected from R₄, H, Alkyl, Cycloalkyl, Aryl, a 5-membered heteroaromatic ring system, or a 6-membered heteroaromatic ring system;
R₃ is H or when R₂ is R₄, R₃ is a covalent bond to R₂ to form a ring structure;
R₄ is a C1-C6 alkylene moiety substituted with at least one O-Alkyl, S-Alkyl or OH substituent;
each of W and X independently is CH₂ and S, provided that W and X are not both CH₂;
Alkyl is a C1-C6 straight or C4-C8 branched chain alkyl group optionally having at least one hydroxyl substituent;
Aryl is an unsubstituted or substituted phenyl group; and
Cycloalkyl is a C3-C6 cycloalkyl group optionally having at least one hydroxyl substituent;
wherein the R₂ and Aryl groups in formula (II) may optionally comprise substituent moieties selected from fluorine (F), chlorine (Cl), bromine (Br), alkyl, hydroxy (OH), alkoxy (O-alkyl) and alkylthio (S-alkyl)
with the provisos that:
when R₂ in formula (II) is H, p is 1, R₃ is H, n is 1, and W and X are both S, then R₁ may not be 4-hydroxyphenyl,
when R₂ in formula (II) is H, p is 0, R₃ is H, n is 1 and W and X are both S, then R₁ may not be 4-hydroxyphenyl, and
a compound of formula (II) may not be [1-β-Mpa,7-Sar]OT or deamino[7-glycine] oxytocin; or
(c) carbetocin.

49. A pharmaceutical composition comprising oxytocin or an oxytocin receptor agonist made by the method of claim 32.

50. A method of treating a neurological disorder or reproductive disorder, comprising administering to a subject in need thereof oxytocin or an oxytocin receptor agonist made by the method of claim 32.

51. The method of claim 50, wherein the method is for treating Prader-Willi syndrome; treating or reducing the risks of uterine atony following vaginal delivery, delivery by Caesarean section, or in a patient who is at risk of developing postpartum haemorrhage (PPH); or treating or reducing the risks of excessive bleeding following vaginal delivery.
